SEQUENCE LISTING (10 sequences)

SEQ ID NO: 1 - DNA, 24 bp, Artificial Sequence
  Synthetic primer for Real-Time PCR
gtggcagtgaagaccatgaagttg

SEQ ID NO: 2 - DNA, 22 bp, Artificial Sequence
  Synthetic primer for Real-Time PCR
gaactccgggatagggagtcat

SEQ ID NO: 3 - DNA, 20 bp, Artificial Sequence
  Synthetic primer for Real-Time PCR
gacgccatcaaggtctactg

SEQ ID NO: 4 - DNA, 18 bp, Artificial Sequence
  Synthetic primer for Real-Time PCR
acgggaatccatcggtca

SEQ ID NO: 5 - DNA, 22 bp, Artificial Sequence
  Synthetic primer for Real-Time PCR
gcaagaaggacaaccgaggaaa

SEQ ID NO: 6 - DNA, 22 bp, Artificial Sequence
  Synthetic primer for Real-Time PCR
ggacatcagtgaaggagccaga

SEQ ID NO: 7 - DNA, 21 bp, Artificial Sequence
  Synthetic primer for Real-Time PCR
gctgttatgggtgaaactctg

SEQ ID NO: 8 - DNA, 23 bp, Artificial Sequence
  Synthetic primer for Real-Time PCR
gaataataaggtggagatgcagg

SEQ ID NO: 9 - DNA, 24 bp, Artificial Sequence
  Synthetic primer for Real-Time PCR
tcctgtggcatccacgaaactaca

SEQ ID NO: 10 - DNA, 24 bp, Artificial Sequence
  Synthetic primer for Real-Time PCR
accagacagcactgtgttggcata

(19) United States
(12) Patent Application Publication
(10) Pub. No.: US 2022/0193126 A1
(43) Pub. Date: Jun. 23, 2022

(54) METHODS OF TREATING IMBALANCE OF PLASMA LIPIDS LEVEL AND FATTY LIVER WITH M2C MACROPHAGES

(71) Applicant: National Pingtung University of Science and Technology, Neipu (TW)

(72) Inventor: Ko-Tung CHANG, Neipu (TW)

(21) Appl. No.: 17/545,614

(22) Filed: Dec. 8, 2021

(30) Foreign Application Priority Data
Dec. 18, 2020 (TW) 109145113

(51) Int. Cl. A61K 35/15; A61K 38/45; A61P 1/16

(52) U.S. Cl. CPC A61K 35/15; A61P 1/16; A61K 38/45

(57) ABSTRACT

The present invention discloses methods of treating imbalance of plasma lipids level, fatty liver, nephritis or vascular fibrosis with M2C macrophages. The M2C macrophages used in said methods is prepared by isolating mononuclear cells from bone marrow or peripheral blood, then inducing the mononuclear cells to differentiate into M2 macrophages by macrophage colony stimulating factor (M-CSF), and then inducing the M2 macrophages to polarize into MERTK-expressing M2C macrophages by baicalin. The treatment of imbalance of plasma lipids level comprises increasing proportion of high-density lipoprotein cholesterol in plasma lipids. The invention also provides methods of treating fatty liver and nephritis in a subject in need, comprising the step of administering a therapeutically effective amount of MERTK-expressing M2C macrophages.

### CROSS-REFERENCE TO RELATED APPLICATIONS

This application claims the benefit of priority of Taiwan Application Serial Number TW109145113, filed Dec. 18, 2020, which is herein incorporated by reference in its entirety.

### TECHNICAL FIELD

This disclosure is related to methods of treating imbalance of plasma lipids level, fatty liver, nephritis or vascular fibrosis with M2C macrophages. The M2C macrophages used in said methods are MERTK-expressing M2C macrophages prepared by isolating mononuclear cells from bone marrow or peripheral blood, then inducing the mononuclear cells to differentiate into M2 macrophages by macrophage colony stimulating factor (M-CSF), and then inducing the M2 macrophages to polarize into MERTK-expressing M2C macrophages by baicalin.

### BACKGROUND

Taiwan Patent Publication No. TWI661046B describes M2C macrophages and application thereof. The M2C macrophages is prepared by isolating mononuclear cells from bone marrow or peripheral blood, then inducing the mononuclear cells to differentiate into M2 macrophages by macrophage colony stimulating factor (M-CSF), and inducing the M2 macrophages to polarize into M2C macrophages by baicalin. In Taiwan Patent Publication No. TWI661046B, the M2C macrophages obtained express high level of Mer tyrosine kinase (MERTK), pentraxin 3 (PTX3), and programmed cell death ligand 1 (PD-L1) expression and exert high phagocytosis and anti-inflammatory activity. However, prior art does not describe or teach whether the M2C macrophages induced by baicalin can reduce other diseases in mammalian.

### SUMMARY

The inventor conducted deep research on the baicalin induced M2C macrophages cell line to obtain the use of the cell line besides treating chronic inflammatory disease or autoimmune disease.

The inventor found that treating high-fat diet-fed hyperlipidemias mouse model with the baicalin induced M2C macrophages can lower the plasma lipids level in mice and reduce fatty liver and nephritis. The inventor also found treatment with baicalin induced M2C macrophages can decrease the non-alcoholic fatty liver disease (NAFLD) associated genes expression in liver. Therefore, the present invention provides the following technical means:

A method of treating imbalance of plasma lipids level in a subject in need, comprising the step of administering a therapeutically effective amount of M2C macrophages, wherein the M2C macrophages is MERTK-expressing M2C macrophages prepared by:

isolating mononuclear cells from bone marrow or peripheral blood;

inducing the mononuclear cells to differentiate into M2 macrophages by macrophage colony stimulating factor (M-CSF); and

inducing the M2 macrophages to polarize into MERTK-expressing M2C macrophages by baicalin.

In an embodiment, the treatment of imbalance of plasma lipids level comprises increasing proportion of high-density lipoprotein cholesterol in plasma lipids.

In an embodiment, the M2C macrophages are macrophage cell lines deposited in CCTCC (China Center for Type Culture Collection) with accession number C2017269.

The present invention also provides a method of treating at least one condition selected from the group consisting of fatty liver, nephritis and vascular fibrosis in a subject in need, comprising the step of administering a therapeutically effective amount of M2C macrophages, wherein the M2C macrophages is MERTK-expressing M2C macrophages prepared by:

isolating mononuclear cells from bone marrow or peripheral blood;

inducing the mononuclear cells to differentiate into M2 macrophages by macrophage colony stimulating factor (M-CSF); and

inducing the M2 macrophages to polarize into MERTK-expressing M2C macrophages by baicalin.

In an embodiment, the M2C macrophages are macrophage cell lines deposited in CCTCC (China Center for Type Culture Collection) with accession number C2017269.

In an embodiment, the treatment of fatty liver comprises at least one of reducing microvesicular fatty liver and reducing bridging fibrosis of liver.

In an embodiment, the treatment of fatty liver disease comprises downregulation of non-alcoholic fatty liver disease associated genes in the liver, and the non-alcoholic fatty liver disease associated genes is one or more selected from the group consisting of collagen type 1 alpha (COL1A1), fibronectin (FN), peroxisome proliferator-activated receptor-gamma (PPARγ).

In an embodiment, the treatment of nephritis comprises at least one of reducing arterial wall thickening at renal glomerulus and lessening arterial onion skin lesion at renal glomerulus.

In the specification and the drawings of the present invention, the M2C macrophages induced by baicalin are also mentioned briefly as MERTK^{+/hi} M2 cells.

### BRIEF DESCRIPTION OF THE DRAWINGS

FIG. 1 illustrates that the flow chart of establishment of hyperlipidemias mouse model, cell injections and timing of sampling in Example 3.

FIG. 2A shows the photograph of the mice in the control group in Example 3;

FIG. 2B shows the photograph of the mice in the Ath group in Example 3; FIG. 2C shows the photograph of the mice in the Ath+M2 group in Example 3.

FIGS. 3A and 3B show the microscope images which demonstrate the morphological difference in between the placebo-treated macrophages (FIG. 3A) and baicalin-treated macrophages (FIG. 3B) in Example 1.

FIGS. 4A, 4B, 4C, 4D and 4E illustrate the results of flow cytometry analysis in Example 2. FIG. 4A and FIG. 4B illustrate the scatter diagram of CD11b and MERTK expression on macrophages; FIG. 4C illustrates the bar chart showing the proportion of cells expressing CD11b and MERTK; FIG. 4D illustrates the comparison of MERTK protein expression level; FIG. 4E illustrates the comparison of average MERTK protein expression level in single cell.

FIGS. 5A, 5B and 5C show the photographs of the appearance of organs in mice of each group after 7 weeks of experiment in Example 3.

FIG. 6 illustrates the bar chart showing the change of average weights of mice in each group during test in Example 3.

FIG. 7A illustrates the result of plasma cholesterol level measurement in Example 4; FIG. 7B illustrates the result of plasma triglyceride level measurement in Example 4.

FIG. 8 shows the photographs of the appearance of hearts in mice of each group after 7 weeks of experiment in Example 3.

FIG. 9A, FIG. 9B and FIG. 9C show the H&E staining images of cross sections of heart tissue in mice of each group in Example 5.

FIG. 10 shows the Masson's trichrome staining images of cross sections of heart tissue in mice of each group in Example 5.

FIG. 11 shows the photographs of the appearance of aorta tissue in mice of each group after 7 weeks of experiment in Example 3.

FIG. 12A, FIG. 12B, FIG. 12C and FIG. 12D show the H&E staining images of cross sections of aorta tissue in mice of each group in Example 5.

FIG. 13 shows the photographs of the appearance of liver tissue in mice of each group after 7 weeks of experiment in Example 3.

FIG. 14 illustrates the bar chart showing the liver weight of mice of each group in Example 3.

FIG. 15 shows the H&E staining images of cross sections of liver tissue in mice of each group in Example 6.

FIG. 16 shows the Masson's trichrome staining images of cross sections of liver tissue in mice of each group in Example 6. The letter “CV” indicates the central vein, and the letter “PV” indicates the portal vein. Scale bar: 100 μm, magnification 100×.

FIG. 17 shows the photographs of the appearance of kidney tissue in mice of each group after 7 weeks of experiment in Example 3.

FIG. 18A and FIG. 18B show the H&E staining images of cross sections of kidney tissue in mice of each group in Example 7.

FIG. 19 shows the hepatic relative gene expression level of MERTK, COl1A1, FN, PPARγ in mice of each group in Example 8. The relative gene expression of target genes was normalized to β-actin. a: p<0.05 compared with the normal diet group; b: p<0.05 compared with the Ath diet group. Data are presented as mean±SEM.

FIG. 20 is a graphical illustration of the possible mechanism of alleviation of NAFLD with baicalin induced MERTK^{+/hi} M2 macrophages.

### DETAILED DESCRIPTION

The embodiments disclosed below are intended to describe the details and the effect of the present invention, not to limit the present invention to be put into practice in the way described below.

### EXAMPLES

### Example 1: Induction of M2C Macrophage Polarization

The bone marrow was flushed out from the femurs of mice (C57BL/6J, 8-week-old to 20-week-old, from National Laboratory Animal Center in Taipei, Taiwan) with phosphate-buffered saline (PBS) containing BSA 0.5%. The peripheral blood or bone marrow flushing fluid were layered with Ficoll-Hypaque (GE Healthcare, Uppsala, Sweden) and centrifuged at 400×g for 40 minutes at 18° C. to isolate the mononuclear cell of peripheral blood and bone marrow. The interphase layer containing mononuclear cell was washed with PBS and centrifuged for 10 minutes at 100×g, 18° C. twice. Plate mononuclear cells in dish culture with RPM 1640 supplemented with 10% FBS (fetal bovine serum) and 50 ng/ml recombinant mouse M-CSF (PeproTech, British) at 37° C. and 5% CO₂ for 7 days to induce the mononuclear cells to differentiate into macrophage. On the next day of the 7^{th} day of M-CSF treatment, 5×10⁵ cells of bone marrow derived macrophages (BMDM) cells were plated in RPMI 1640 (10% FBS) and incubated overnight. After overnight incubation, the BMDM cells were incubated in RPMI 1640 (10% FBS) supplemented with 50 μM Baicalin for 24 hours to induce the BMDM cells to polarize into M2C macrophage.

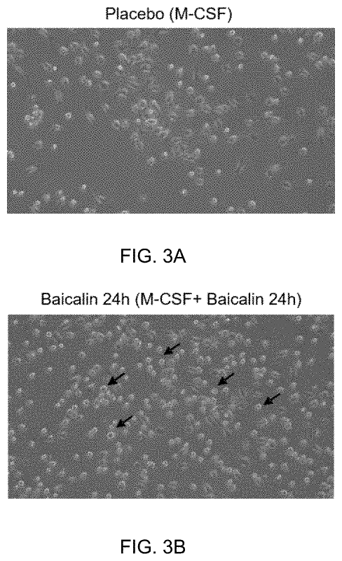

The microscope images (FIG. 3A and FIG. 3B) showed that the morphology of the M2C macrophages induced by baicalin (cells indicated by arrow in FIG. 3B) was basically round shape, rather than spindle shape as placebo-treated group (M-CSF) (FIG. 3A). The result showed the morphology of the M2C macrophages induced by baicalin was significantly different from typical M2C macrophages.

### Example 2: Confirmation of MERTK Expression on M2C Macrophages

1×10⁶ cells of the M2C macrophages induced by baicalin were analyzed by flow cytometry to confirm whether the M2C macrophages express MERTK. Fluorescence-labelled anti-mouse antibodies (Biolegend, the U.S.A.), FITC-anti-CD 11b and PE-anti-MERTK, are used in the analysis. Cells in centrifuge tube reacted with 1-2 μl antibodies on ice for 30 minutes protected from light and then were centrifuged at 300×g, 4° C. for 5 minutes. After removing supernatant, the sample was dissolved in 1 ml of PBS and analyzed by flow cytometry.

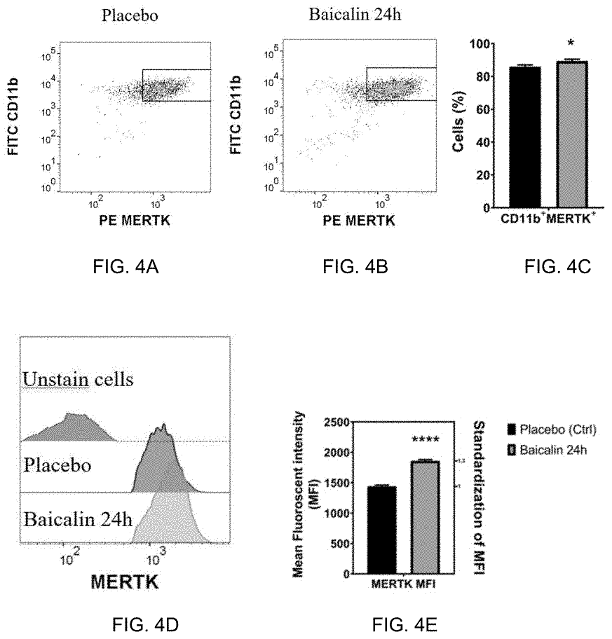

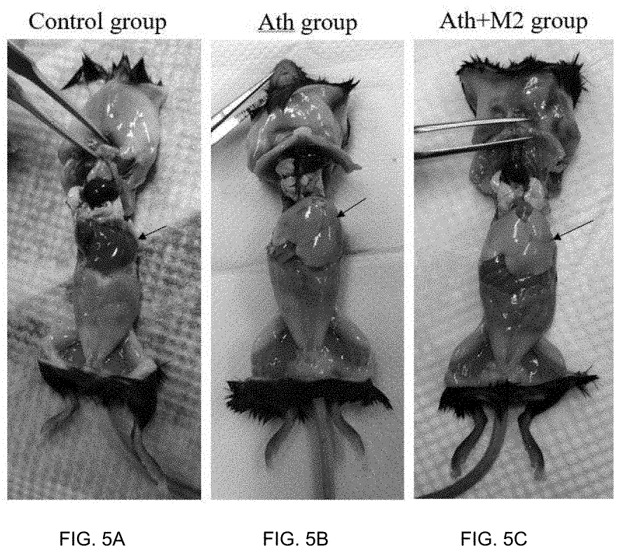

As the analysis result showed in FIG. 4A, after immunostaining, fluorescent label combined with MERTK was detected on a certain proportion of cells in both placebo-treated group and baicalin-treated group. However, when the inventor used mean fluorescence intensity (MFI) to evaluate average MERTK expression level on single cell, as showed in FIG. 4D, the MERTK expression level on the M2C macrophages induced by baicalin was significantly enhanced. In the specification and the drawings of the present invention, the M2C macrophages induced by baicalin are also mentioned briefly as MERTK^{+/hi} M2 cells.

### Example 3: Establishment of Hyperlipidemias Mouse Model

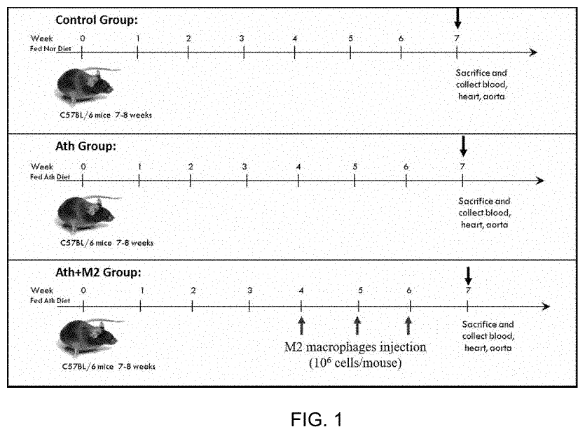

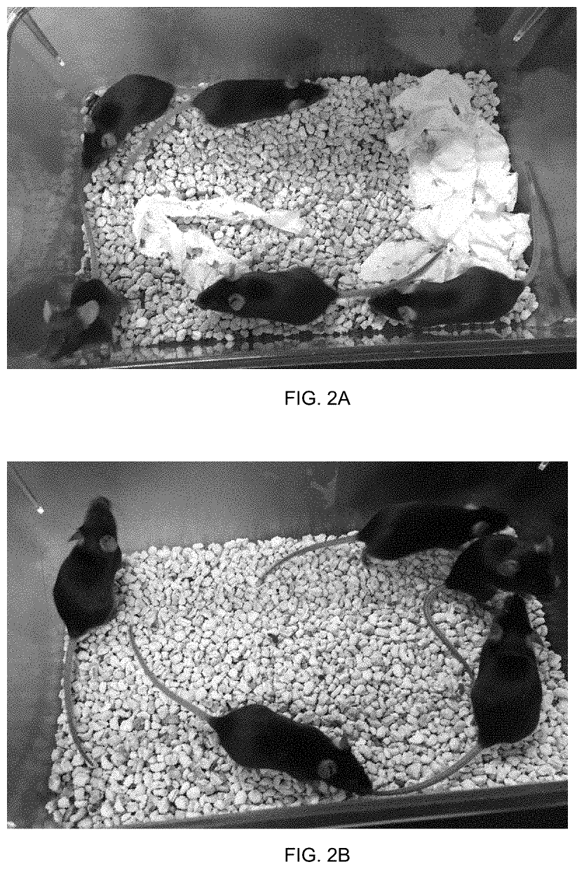

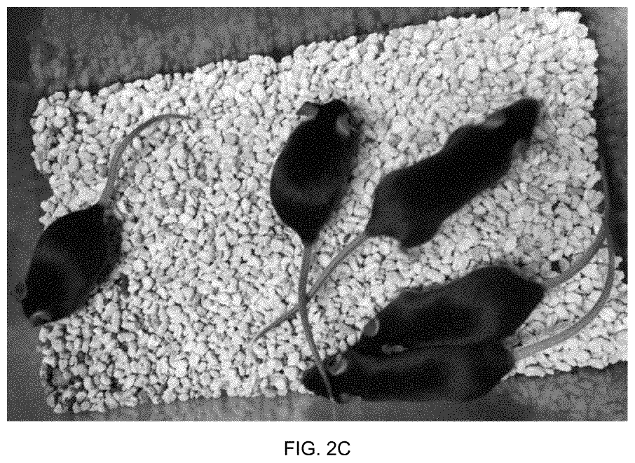

To evaluate the effect of treatment by MERTK^{+/hi} M2 cells in mammalian, hyperlipidemias mouse model was established to evaluate the effect of MERTK^{+/hi} M2 cells injection in the mouse model. All the test procedures were approved by the Institutional Animal Care and Use Committee at National Pingtung University of Science and Technology in Taiwan. 7-week-old to 8-week-old C57BL/6 mice were housed in a clean conventional animal facility at 22 Celsius degrees with 12 hours light/dark cycle and given food and water ad libitum. The mice were randomly divided into a control group (given normal diet), an Atherosclerosis group (Ath group) (given high fat diet) and an Ath+MERTK^{+/hi} M2 group (Ath+M2 group) (given high fat diet and MERTK^{+/hi} M2 cells injections). Each group comprised ten mice. The experimental design for each group is illustrated in FIG. 1. The mice were fed normal diet or high fat diet (TestDiets, the U.S.A.) for 7 weeks, wherein the Ath+M2 group received retro-orbital MERTK^{+/hi} M2 cells injection at the 4^{th}, 5^{th} and 6^{th} week, that was three times in total. Weight of mice was measured and recorded after feeding on each week. Mice were sacrificed after 7 week and heart, aorta, peripheral blood, bone marrow, liver, and kidney were collected. Tissue samples from different mice were numbered from 1 to 10.

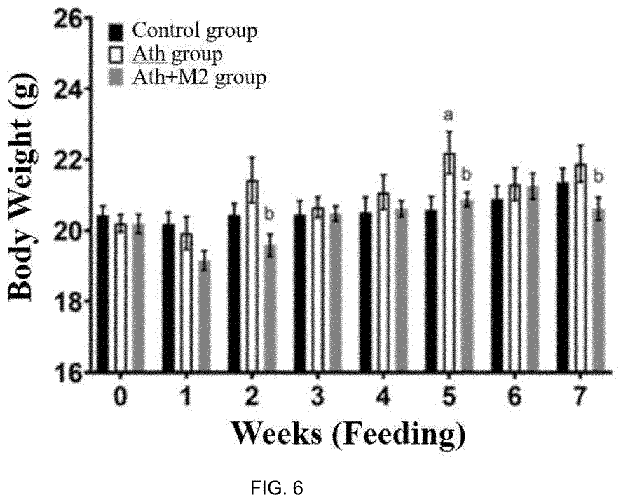

The results of weight analysis are shown in FIG. 6. Representative data are shown by mean±SEM, n=10. *: significantly different compared with the control group, p<0.05. ***: significantly different compared with the control group, p<0.001. The body weights of the Ath+M2 group were reduced significantly compared with the Ath group after injection with MERTK^{+/hi} M2 cells for 3 times.

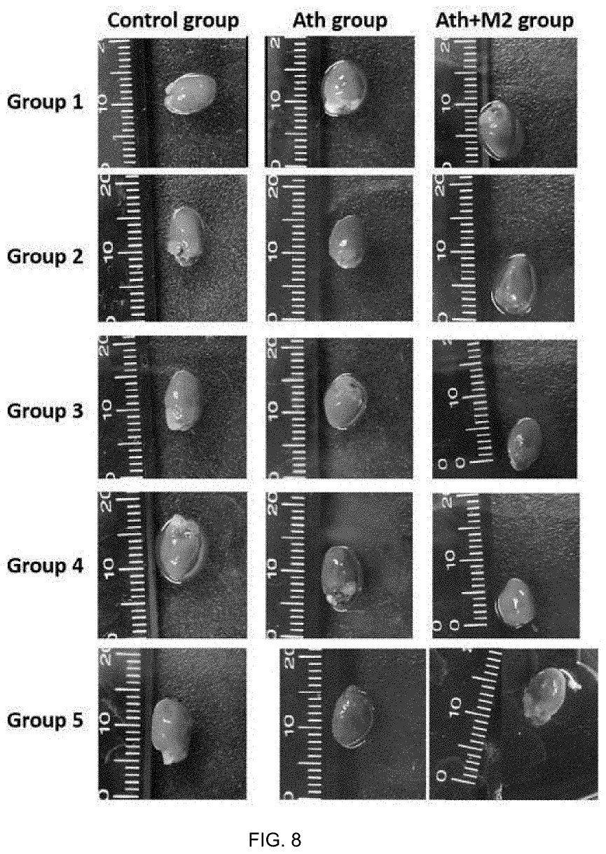

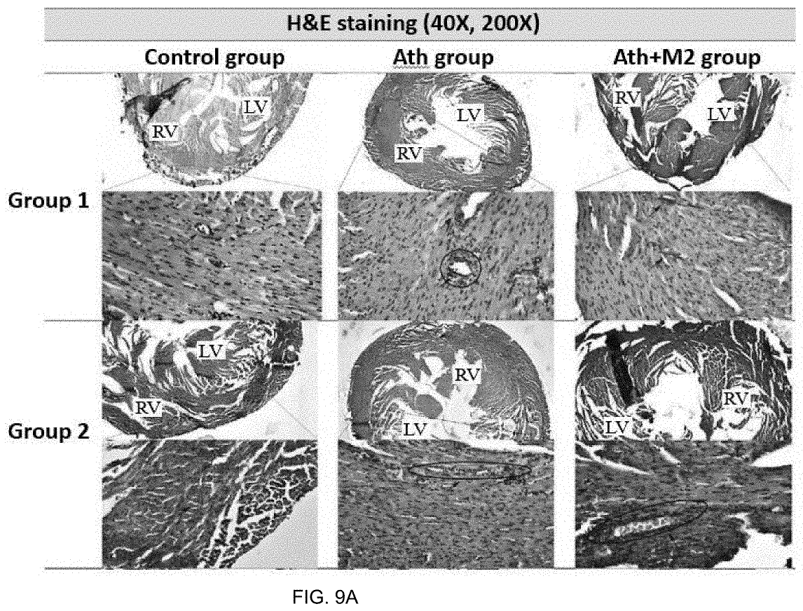

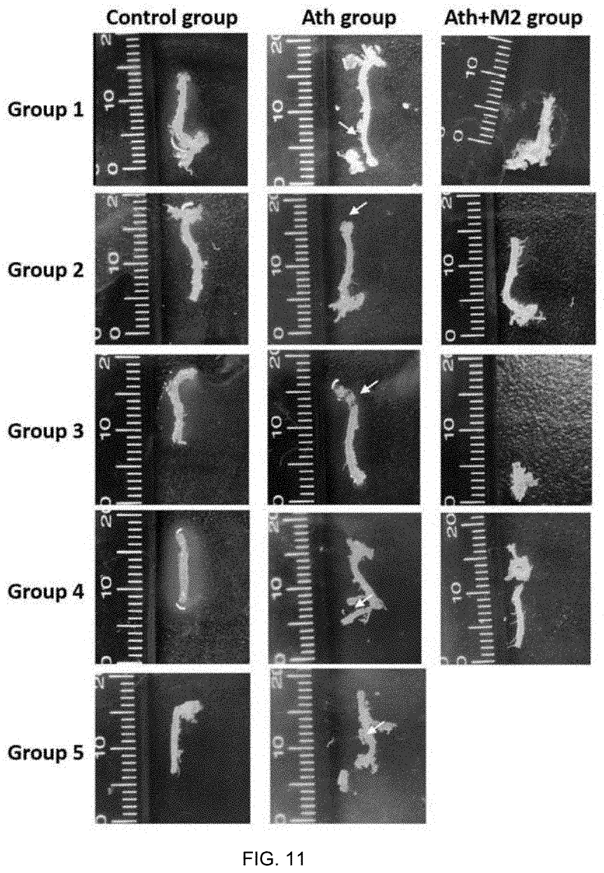

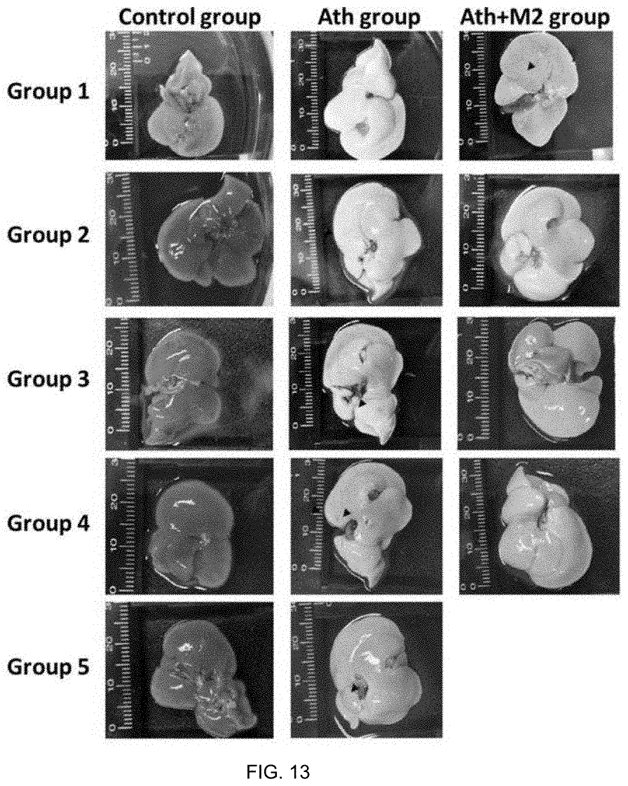

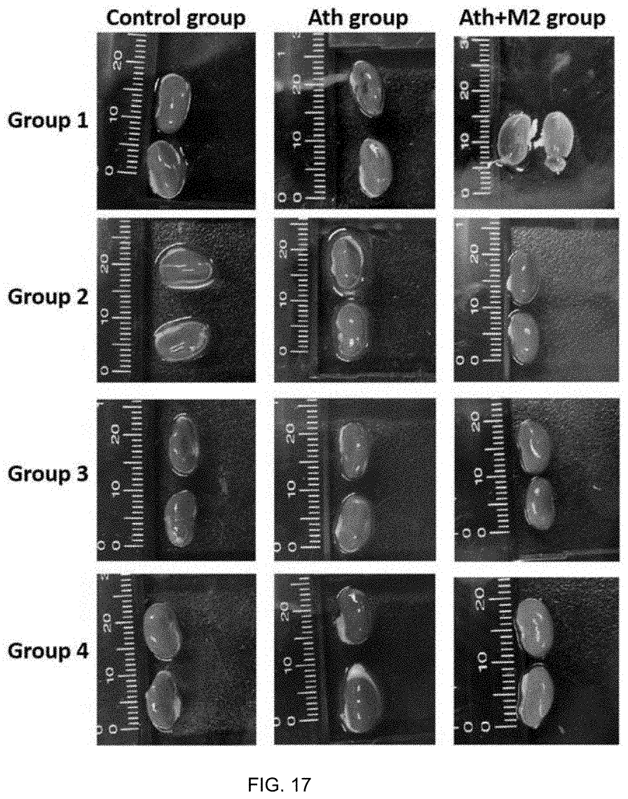

The morphology of heart tissue is shown in FIG. 8. The appearance of hearts had not significant difference between each group. The morphology of aorta is shown in FIG. 11. Compared with the control group, the aorta of the Ath group showed some blood streaks (as white arrow indicated). However, the aorta of the Ath+M2 group showed less blood streak in comparison with that of the Ath group. The morphology of kidneys tissue is shown in FIG. 17. The appearance of kidneys had not significant difference between each group. The morphology of liver tissue is shown in FIG. 13. The livers from the control group were red, smooth and shiny liver tissues. The livers from the Ath group were extremely pale, enlarged, and with blood streak (as black arrow indicated). The livers of the Ath+M2 group received high-fat diet for 7 weeks with 3 times of MERTK^{+/hi} M2 cells injection were a little bit pale and with less blood streak compared with the Ath group. The results indicated that MERTK^{+/hi} M2 cells injection lessened the liver lesions in mice.

### Example 4: Evaluation of Plasma Lipids Composition

Mice were fasted overnight before blood sample collection through retro-orbital veins under isoflurane anesthesia. Heparin or EDTA was used as the anticoagulant. The blood and anticoagulant were mixed for 20 minutes and the mixture were centrifuged for 30 min at 2-8° C., 3000 rpm. Plasma was collected in the supernatant. For eliminating platelet effect, further centrifugation was performed for 10 min at 2-8° C., 10000 rpm as suggested. The cholesterol and triglyceride level in the plasma were measured with Cholesterol Assay Kit (HDL and LDL/VLDL, ab65390, Abcam) and Triglyceride Assay Kit (ab65336, Abcam).

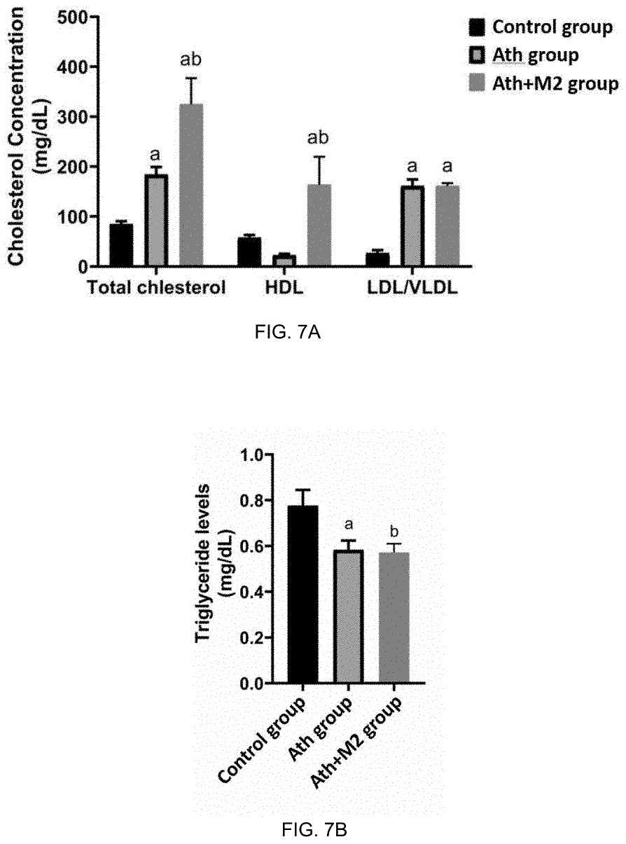

The results of measurement of plasma cholesterol level are shown in FIG. 7A. Representative data are shown by mean±SEM, n=10. The label “a” in the figure means being significantly different with the control group, p<0.05. The label “b” in the figure means being significantly different with the Ath group, p<0.05. The results of measurement of plasma triglyceride level are shown in FIG. 7B. Representative data are shown by mean±SEM, n=10. The label “a” in the figure means being significantly different with the control group, p<0.0001. The label “b” in the figure means being significantly different with the control group, p<0.001.

In the results of above plasma lipids analysis, compared with the control group, the total cholesterol level and the Low-density lipoprotein/Very low-density lipoproteins (LDL/vLDL) cholesterol (harmful cholesterol) level significantly increased in the Ath group mice. Compared with the Ath group, the high-density lipoprotein (HDL) cholesterol (beneficial cholesterol) level of Ath+M2 group was significantly increased. Compared with the control group, the triglyceride level decreased in both the Ath group mice and the Ath+M2 group. The results suggested that MERTK^{+/hi} M2 cells injection reduced the imbalance of plasma lipids level in the mice fed with high fat diet, especially it increased the proportion of HDL cholesterol level in plasma lipids.

### Example 5: Histological Analysis of Heart and Aorta

The heart and aortic tissues were embedded in optimal cutting temperature compound. The samples were then perfused with saline and then with 10% formalin. After overnight treatment, the samples were washed with ddH2O and immersed in the respective fixative (70%, 80%, 90%, 95%, 100% ethanol) for an hour. Then the samples were placed in xylene for an hour and repeated for three times. After dried, the samples were embedded in paraffin. Cryosections of the sample with thickness 5-10 μm were stained with haematoxylin and eosin (H&E staining) or Masson's trichrome staining.

H&E staining: Paraffin sections were flatten in water bath, then sticked to glass slides, and then air dried. After air drying, the samples were soaked in xylene for 5 minutes and repeat three times to dewax. Then, the samples were soaked in 100% ethanol for one minute and repeat twice. Samples were further soaked in 95% ethanol for 30 seconds, and placed in water for 30 seconds. Excess water on the slides was removed, and slides were stained with hematoxylin for 4 minutes. After staining, slides were rinsed under running water for 10 minutes. Excess water was removed on the slides and slides were stained with eosin for 10 seconds. In the following dehydrating step, slides were soaked in 95% ethanol for one minute twice, 100% ethanol for 2 minutes, xylene for 5 minutes and then xylene for 10 minutes. After air drying, the slides were observed under a microscope.

Masson's trichrome staining: Trichrome staining (TRM-2-IFU, ScyTek, the U.S.A.) was used. The sections were deparaffinized. Bouin's Fluid was pre-heated in a water bath to 56-64° C. in a fume hood or ventilated area. 5-10 drops of preheated Bouin's Fluid were applied to tissue section and stood for 60 minutes. Slides were rinsed in tap water and then further rinsed once in distilled water. Equal parts of Weigert's (A) and Weigert's (B) were mixed and the mixture (working Weigert's Iron Hematoxylin) was used to stain the slides for 5 minutes. During staining, sufficient amount of stain were maintained to prevent stain drying on slide. Next, the slides were rinsed in running tap water for 2 minutes. Biebrich Scarlet and Acid Fuchsin Solution were applied to the slides and stood for 15 minutes. Next, the slides were rinsed in distilled water. The slides were treated with Phosphomolybdic/Phosphotungstic Acid Solution for 10-15 minutes or longer until collagen is not red. Aniline Blue Solution was applied to the slides and stood for 5-10 minutes. The slides were rinsed in distilled water. Acetic Acid Solution (1%) was applied to the slides and stood for 3-5 minutes. Then the samples were dehydrated through treating with 95% Alcohol twice and followed by treating with Absolute Alcohol twice. Dehydrating agent were removed with xylene, and samples were mounted in synthetic resin afterward.

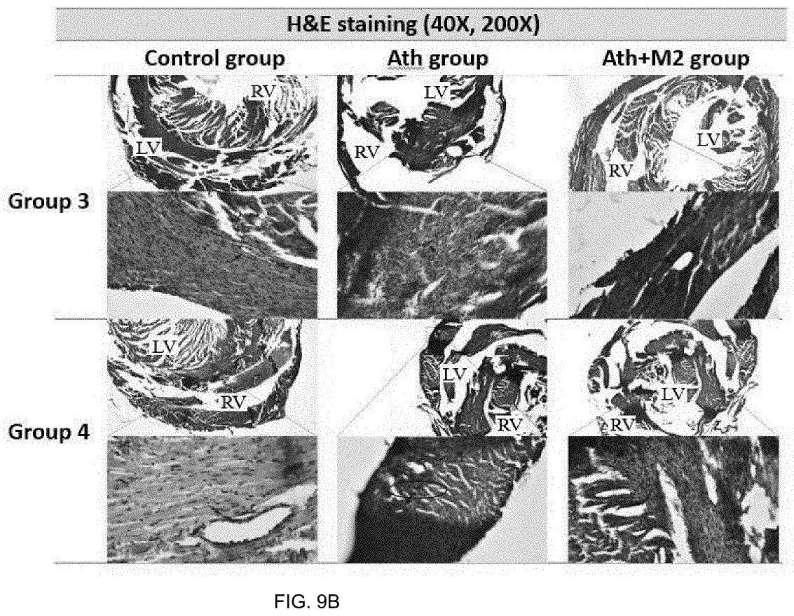

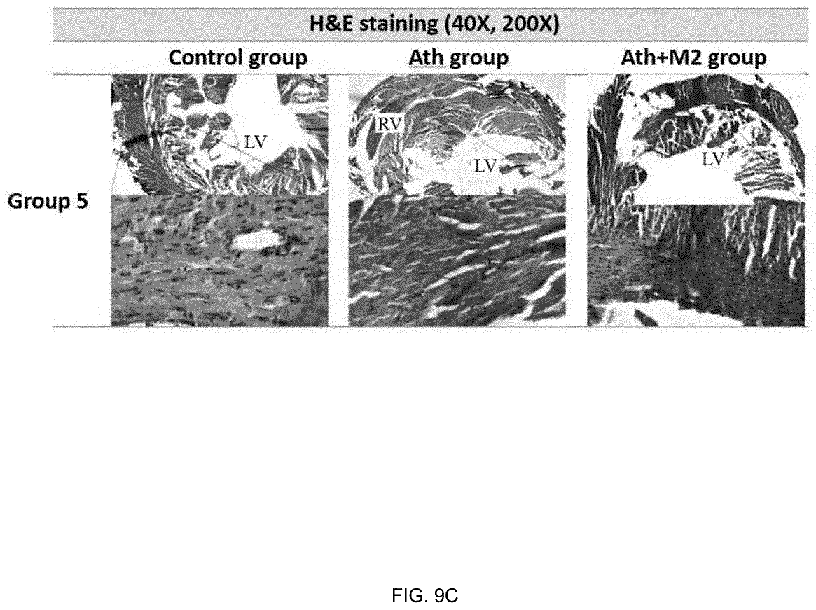

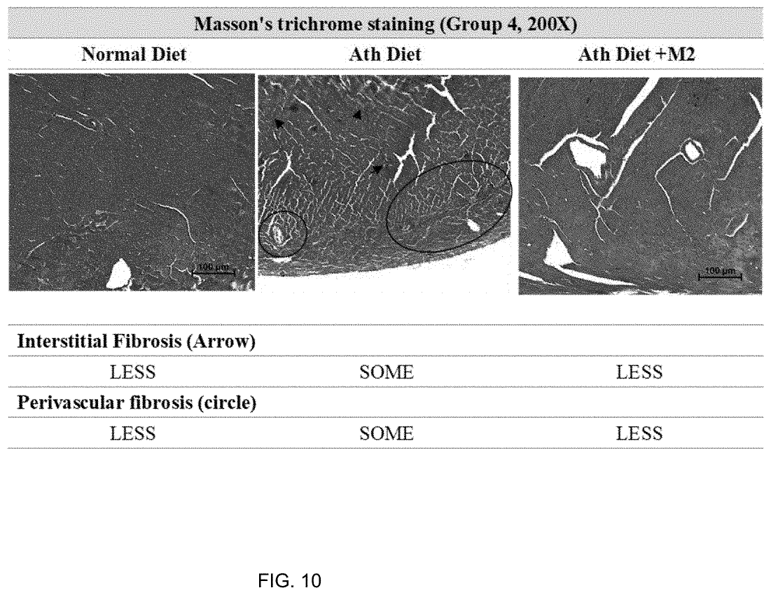

In the results of H&E staining, Group 1 to 5 were tissue slices from different mice. The inventor observed that some coronary walls showed increased thickness (as the black circles indicate in FIG. 9A, FIG. 9B, FIG. 9C) and increased immune cells infiltrations (as the arrows indicate in FIG. 9A, FIG. 9B, FIG. 9C) in the heart of the Ath group, compared to the control group and the Ath+M2 group. In the results of Masson's trichrome staining, the inventor observed the representative sections of myocardium from Group 4 mouse and found interstitial fibrosis (as the arrow indicates in FIG. 10) and a trend of perivascular fibrosis (as the circle indicates in FIG. 10) in the Ath group. However, compared to the Ath group, the fibrosis was alleviated in the mouse from the Ath+M2 group.

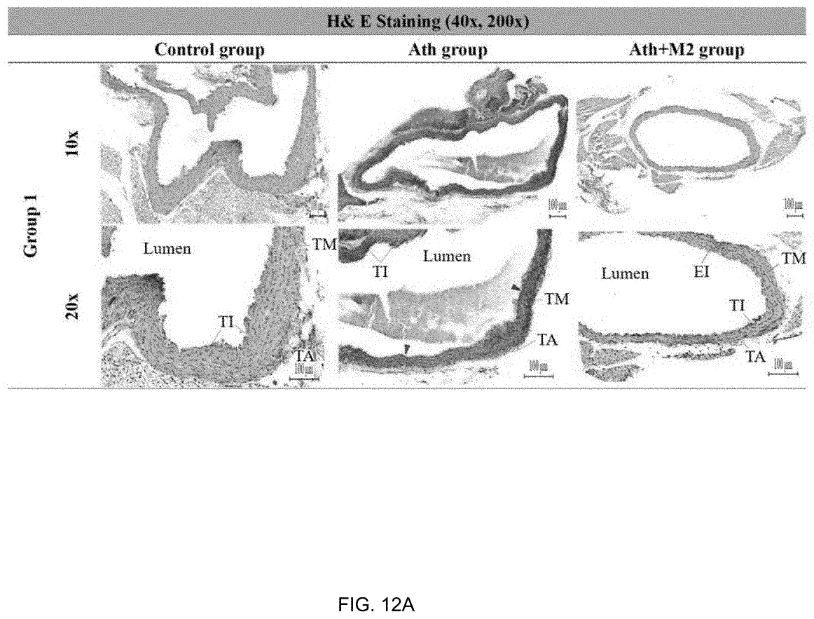

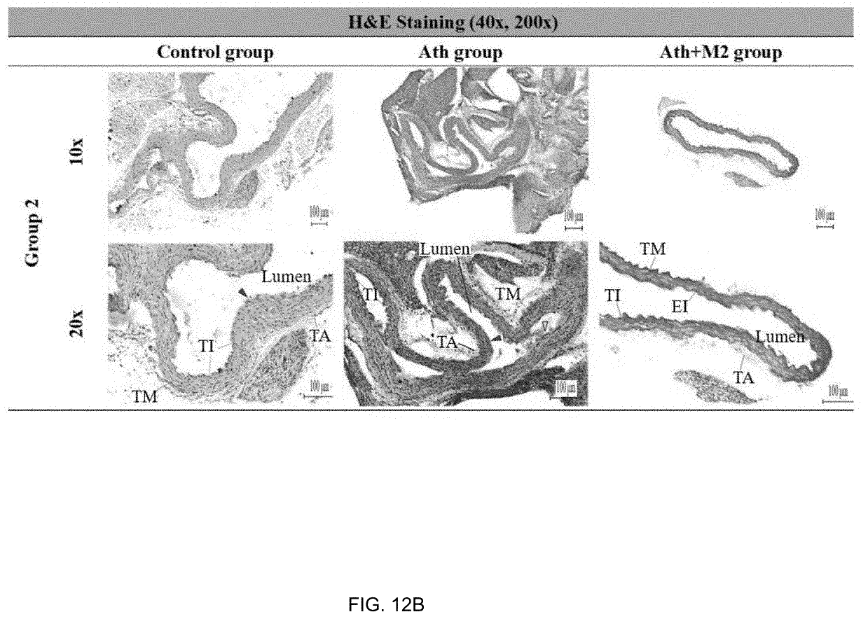

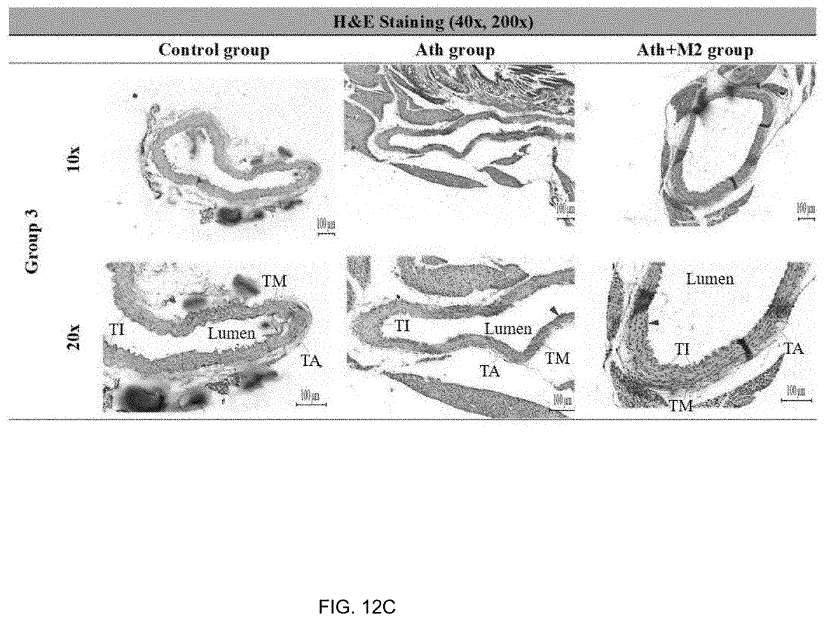

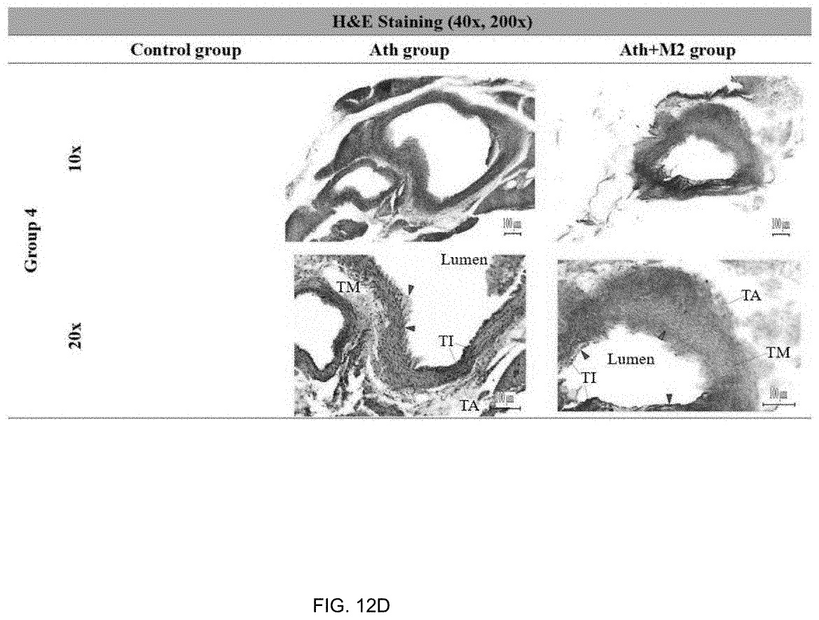

H&E staining of the aorta tissues are shown in FIG. 12A, FIG. 12B, FIG. 12C and FIG. 12D. In FIG. 12A, FIG. 12B, FIG. 12C and FIG. 12D, TI means Tunica Intima; TM means Tunica Media; and TA means Tunica Adventitia. Aortic vascular smooth muscle cells and elastic fibers were stained red and collagen fibers were stained blue-green. The arrows in red, yellow, blue, green and black in FIG. 12 indicate different stage of progression of Atherosclerosis respectively, as shown in Table 1. The analysis results of Atherosclerosis in FIG. 12 are shown in Table 1.

TABLE 1

| | | | Ath + M2 |
|---|---|---|---|
| Group | Control group | Ath group | group |
| 1 | — | \textcircled{1}\textcircled{3} | — |
| 2 | \textcircled{1} | \textcircled{1}\textcircled{2} | — |
| 3 | — | \textcircled{1} | \textcircled{1} |
| 4 | | \textcircled{1}\textcircled{3} | \textcircled{1}\textcircled{3}\textcircled{4} |
| Total of | \textcircled{1}: 1/3 (33.3%) | \textcircled{1}: 4/4 (100%) | \textcircled{1}: 2/4 (50%) |
| AI | \textcircled{2}: 0/3 (0%) | \textcircled{2}: 1/4 (25%) | \textcircled{2}: 0/4 (0%) |
| | \textcircled{3}: 0/3 (0%) | \textcircled{3}: 2/4 (50%) | \textcircled{3}: 1/4 (25%) |
| | \textcircled{4}: 0/3 (0%) | \textcircled{4}: 0/4 (0%) | \textcircled{4}: 1/4 (25%) |
| | \textcircled{5}: 0/3 (0%) | \textcircled{5}: 0/4 (0%) | \textcircled{5}: 0/4 (0%) |

Atherosclerosis Indicators (AI) in Arteries
\textcircled{1} Reduction of Tunica Intima Corrugation(red arrow)
\textcircled{2} Adhesion of red blood cells on the surface of the intima (yellow arrow)
\textcircled{3} Increase in the thickness of tunica intima (blue arrow)
\textcircled{4} Increase in the thickness of tunica media (green arrow)
\textcircled{5} Presence of Foam Cells (black arrow)

In the results of H&E staining, the control group displayed reduction of tunica intima corrugation (red arrowhead) in Group 2, while the Ath group showed reduction of tunica intima corrugation in all groups. The Ath group showed pathological change like adhesion of red blood cells on the surface of tunica intima in Group 2, and further thickened tunica media was found in Group 1 and Group 4. By contrast, in the Ath+M2 group, though Group 3 and Group 4 still showed reduction of tunica intima corrugation (red arrowhead) but the stains of Group 1 and Group 2 did not show any pathological change.

### Example 6: Histological Analysis of Liver

The weight of liver tissues of mice from each group were measured. 5-10 μm cryosections of the samples were prepared by the same way as Example 5 and stained by H&E staining or Masson's trichrome staining.

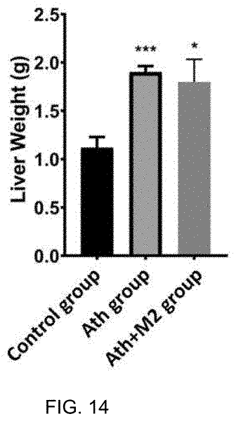

Analysis of the liver weight is shown in FIG. 14. Representative data are shown by mean±SEM, n=4-5. *: significantly different compared with the control group, p<0.05. ***: significantly different compared with the control group, p<0.001. The liver weights of Ath group showed remarkable increase compared to the liver weights of the control group. The increase of liver weights of the Ath+M2 group was relatively less.

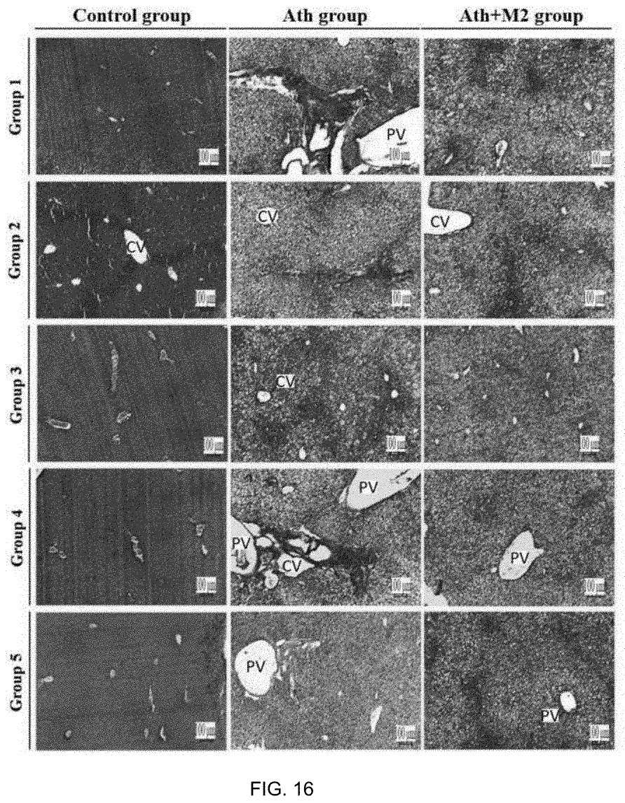

and FIG. 16.

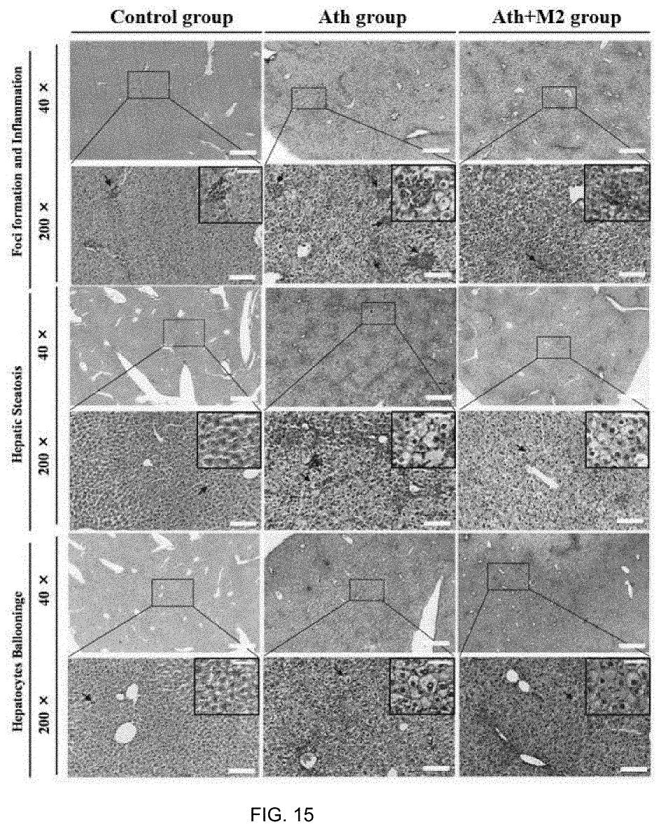

The representative histopathological micrographs of H&E staining and Masson's trichrome staining of each group are shown in FIG. 15 and FIG. 16.

As shown in FIG. 15, 3 major characteristics of non-alcoholic fatty liver disease, foci formation and inflammation, hepatic steatosis, and hepatocyte ballooning, were indicated in the histopathological micrographs. The framed areas are further enlarged in the below 200× micrographs.

The deep purple clusters of heterogeneous inflammation cells, which usually consist of lymphocytes, neutrophils, eosinophils, and macrophages, demonstrated the foci formation and inflammation. Hepatocellular steatosis is classified into two forms: the macrovesicular and the microvesicular. The macrovesicular fatty liver is identified by a single large fat droplet that occupies the cytoplasm of hepatocytes, pushing the nucleus to the periphery, while the microvesicular fatty liver has the cytoplasm of hepatocytes filled with tiny lipid droplets and the nucleus is located centrally in the cells. Hepatocyte ballooning is characterized by swollen hepatocytes and wispy cytoplasm.

The induced histopathological changes was evaluated using a revised Brunt's histological scoring system for NAFLD. 3 to 5 different sections per liver in 5 mice of each group were evaluated and graded with the NAFLD scoring system. This scoring system consists of three major characteristics in the NAFLD liver: hepatic steatosis (S), range from 0-3; inflammation (L), range from 0-3; and the ballooning of hepatocytes (B), range from 0-2. Livers with a <5% steatosis area, no inflammation, and ballooning degeneration, were regarded as 0 scores. Livers with a 5-33% steatosis area, with <2 foci of inflammation/20× field, and few ballooning degenerations are graded as 1. Livers with a >33%-66% steatosis area, with a 2-4 inflammation foci/20× field, with a lot of ballooning degenerations, were scored as 2. Livers with more than 66% of steatosis area and with more than four inflammation foci were scored as 3. The total scores indicate the sum of all the hepatic changes, according to the following equation [Total NAFLD Activity Score (NAS)=S+L+B], and were graded as severe (≥5), moderate (3-4), and mild (<3). To confirm the severity of liver damage, fibrosis and necrosis evaluations were also conducted separately with the range from 0-4. Histological findings, such as mild pericellular fibrosis, moderate pericellular fibrosis, portal/periportal fibrosis without pericellular fibrosis, pericellular and portal fibrosis, bridging necrosis, and cirrhosis are graded as 1a, 1b, 1c, 2, 3 and 4, respectively. The grading is presented as means±SEM (n=5 mice/group) in Table 2 below. ^{a}: p<0.05 compared with normal diet group; ^{b}: p<0.05 compared with Ath diet group.

As shown in the upper 6 photos of FIG. 15, compared to the M2C macrophage treatment group (Ath+M2), multiple focal randomly-distributed aggregates of a heterogenous population of inflammatory cells (foci, indicated with black arrows) were observed in the atherosclerosis group (Ath Diet), demonstrated severe inflammation. As shown in the lower 12 photos, in the Ath Diet group, the larger fat droplets (white spots in micrographs) accumulated in the cytoplasm of swelled hepatocytes with irregular shape. While such severe hepatic steatosis and hepatocytes ballooninge were reduced in the hepatocytes of Ath+M2 group.

In the Masson's trichrome staining (FIG. 16), collagen is stained in blue color, which shows the progression of fibrosis. In the Ath Diet group, fibrosis events across the liver section were revealed, especially around the portal vein (indicated with letter “PV”) and the sinusoidal area. Compared to the Ath Diet group, the collagen deposition around the vessel and sinusoidal area were reduced in the Ath+M2 group.

Table 2 shows the NAFLD activity scores graded as described above. Compared with Ath Diet group, the score of Ath+M2 group decreased significantly in all categories. The NAFLD score evaluation result suggested that treatment with baicalin induced MERTK^{+/hi} M2 macrophages significantly reduced the prevalence of fibrosis in the portal/periportal area, and moderately restricted pericellular fibrosis around the liver bile canaliculi.

TABLE 2
Table 1. NAFLD activity score (NAS) components of the treatment groups.

| | Groups | | |
|---|---|---|---|
| | (Mean ± SEM) | | |
| | | | Ath + M2c |
| Score Components | Control group | Ath Diet group | Group |
| Steatosis | | | |
| Score | 0.00 ± 0.00 | 1.86 ± 0.08 ^{a} | 0.96 ± 0.13 ^{ab} |
| Extent | <5% | >5-33% | >5-33% |
| Inflammation | | | |
| Score | 0.20 ± 0.20 | 2.66 ± 0.38 ^{a} | 1.16 ± 0.24 ^{ab} |
| Extent | None | >4 foci/200x | <2 foci/200x |
| Ballooning | | | |
| degeneration | | | |
| Score | 0.00 ± 0.00 | 2.00 ± 0.00 ^{a} | 1.26 ± 0.12 ^{a} |
| Extent | None | Many | Many |
| Total Scores | 0.20 ± 0.20 | 6.52 ± 0.39 ^{a} | 3.39 ± 0.37 ^{ab} |

Fibrosis Stage

| | | | |
|---|---|---|---|
| Necrosis and Fibrosis | | | |
| Score | 0 | 2 ^{a} | 1b ^{a} |
| Extent | None | Pericellular and | Moderate pericellular |
| | | portal/periportal | fibrosis |
| | | fibrosis | |

### Example 7: Histological Analysis of Kidney

5-10 μm cryosections of kidney tissues of mice from each group were prepared by the same way as Example 5 and stained with H&E staining (as FIG. 17).

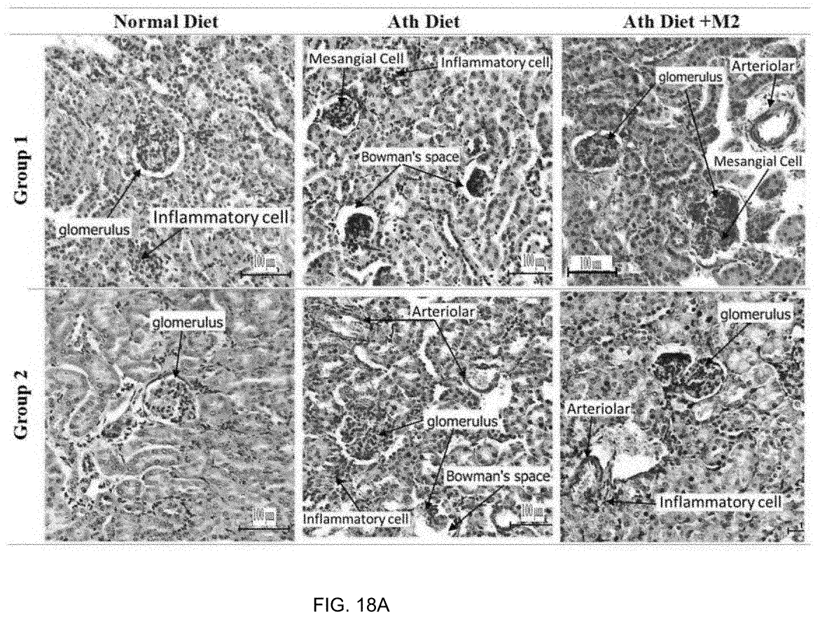

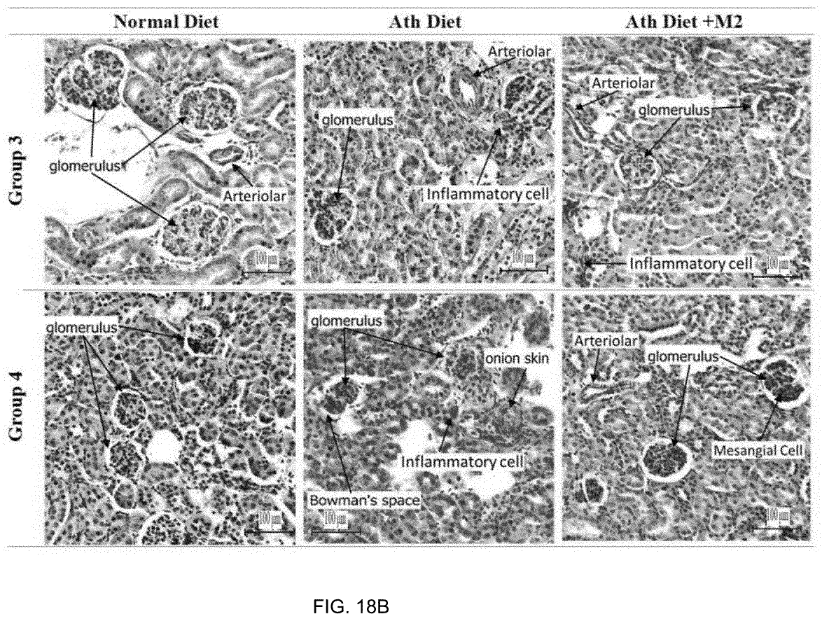

The results of staining are shown in FIG. 18A, FIG. 18B and Table 3. The control group showed normal histology of renal parenchyma, while the kidney of mice from the Ath group showed thickened wall and onion skin lesions, as well as atrophy of glomerular tufts. Compared with the control group, the kidney of the Ath group showed histopathological signs including arteriolar wall thickening, dilatation of the Bowman's Space, mesangial cellular proliferation and glomerular distortion. By contrast, the kidney of the Ath+M2 group did not show dilatation of the Bowman's Space. It implied that MERTK^{+/hi} M2 cells injection may play a role in inhibiting nephritis.

TABLE 3

| | Control | | |
|---|---|---|---|
| Group | group | Ath group | Ath + M2 group |
| 1 | \textcircled{5} | \textcircled{2}\textcircled{3}\textcircled{5} | \textcircled{1}\textcircled{3}\textcircled{4} |
| 2 | | \textcircled{1}\textcircled{2}\textcircled{3}\textcircled{4}\textcircled{5} | \textcircled{1}\textcircled{3}\textcircled{4}\textcircled{5} |
| 3 | | \textcircled{1}\textcircled{2}\textcircled{3}\textcircled{4}\textcircled{5} | \textcircled{5} |
| 4 | \textcircled{3} | \textcircled{1}\textcircled{3}\textcircled{4}\textcircled{5} | \textcircled{2}\textcircled{3}\textcircled{4} |
| 5 | \textcircled{5} | \textcircled{3}\textcircled{4} | |
| Histopathological | \textcircled{1}: 0/5 (0%) | \textcircled{1}: 4/5 (80%) | \textcircled{1}: 2/4 (50%) |
| signs (%) | \textcircled{2}: 0/5 (0%) | \textcircled{2}: 3/5 (60%) | \textcircled{2}: 1/4 (25%) |
| | \textcircled{3}: 1/5 (20%) | \textcircled{3}: 5/5 (100%) | \textcircled{3}: 3/4 (75%) |
| | \textcircled{4}: 0/5 (0%) | \textcircled{4}: 4/5 (80%) | \textcircled{4}: 3/4 (75%) |
| | \textcircled{5}: 2/5 (40%) | \textcircled{5}: 3/5 (60%) | \textcircled{5}: 2/4 (50%) |

Histopathological signs:
\textcircled{1}: Arteriolar wall thickening
\textcircled{2}: Dilatation of the Bowman ′'Space
\textcircled{3}: Mesangial cellular proliferation
\textcircled{4}: Glomerular distortion (lobulation of the tuft)
\textcircled{5}: Inflammatory cells infiltration

### Example 8 Non-Alcoholic Fatty Liver Disease Associated Genes Expression Level Analysis

To evaluate the suppression of non-alcoholic fatty liver disease associated genes by MERTK^{+/hi} M2 cells treatment, the hepatic relative gene expression of MERTK, profibrotic collagen type 1 alpha (COl1A1), profibrotic fibronectin (FN), as well as the lipid metabolism regulator peroxisome proliferator-activated receptor-gamma (PPARγ) were analyzed. The hyperlipidemias mouse model of Example 3 was also used as the non-alcoholic fatty liver disease mouse model (NAFLD mouse model). The total RNA of the liver samples of mice from each group was prepared using Trizol reagent (Invitrogen, Waltham, Mass., USA) according to the manufacturer's protocol. 1 μg of liver total RNA was used in the reverse transcription (RT) reaction by the iScript cDNA Synthesis Kit (Bio-Rad, Hercules, Calif., USA). The quantitative real-time PCR was performed using the KAPA SYBR® FAST qPCR Master Mix (2×) Kit (KAPA Biosystem, Wilmington, Del., USA) according to the manufacturer's protocol. Quantitative PCR reactions were performed using a QIAGEN Rotor Gene Q Real-Time PCR. The primer sequences are shown in Table 4. The relative gene expression of target genes was normalized to β-actin (the housekeeping gene). The constitutive expression of the β-actin gene level was not altered in relation to mice of control group (normal diet). The data were calculated using the standardized mRNA level comparative method 2^{-ΔΔCt}.

TABLE 4

| Target | Primer sequences (5′-3′) | SEQ | |
|---|---|---|---|
| Gene | Forward/Reverse | ID NO. | |
| MERTK | GTGGCAGTGAAGACCATGAAGTTG | 1 | Forward |
| | GAACTCCGGGATAGGGAGTCAT | 2 | Reverse |
| COL1A1 | GACGCCATCAAGGTCTACTG | 3 | Forward |
| | ACGGGAATCCATCGGTCA | 4 | Reverse |
| FN | GCAAGAAGGACAACCGAGGAAA | 5 | Forward |
| | GGACATCAGTGAAGGAGCCAGA | 6 | Reverse |
| PPARγ | GCTGTTATGGGTGAAACTCTG | 7 | Forward |

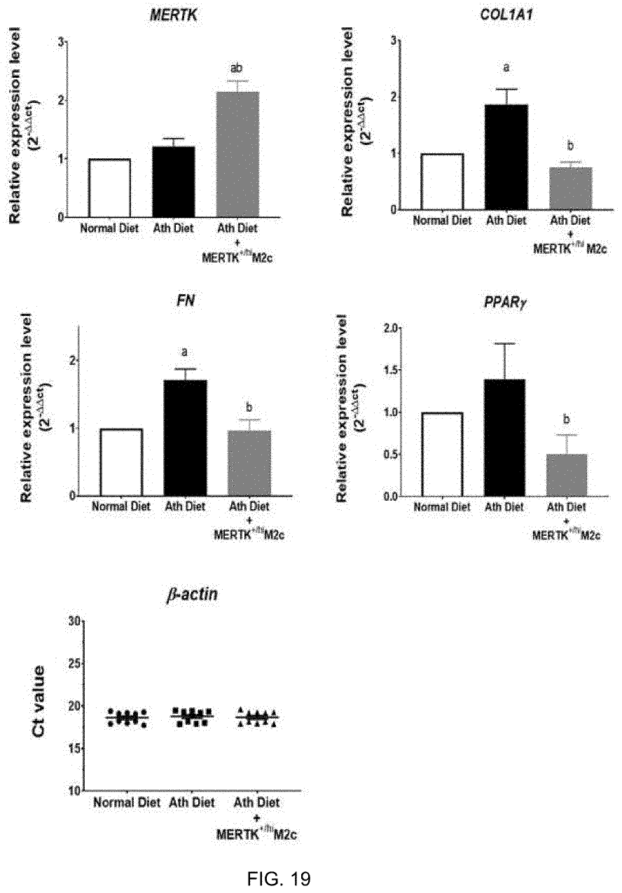

As shown in FIG. 19, the Ath+M2 group (given high fat diet and MERTK^{+/hi} M2 cell treatment group) displayed higher MERTK relative gene expression compared to normal diet control group and Ath Diet group (given high fat diet), suggesting the presence of MERTK^{+/hi} M2 macrophages in the liver. The transcript level of COL1A1, FN and PPARγ were significantly downregulated in mice treated with MERTK^{+/hi} M2 macrophages.

Treatment with MERTK^{+/hi} M2 macrophages downregulated the relative expression of COL1A1 and FN compared with that in the Ath diet group (p<0.05), suggesting a decrease in overall fibrogenesis events. The lipid metabolism related regulator PPARγ was also significantly downregulated in the MERTK^{+/hi} M2 macrophages treatment group compared with the Ath diet group, suggesting that fat accumulation was reduced significantly.

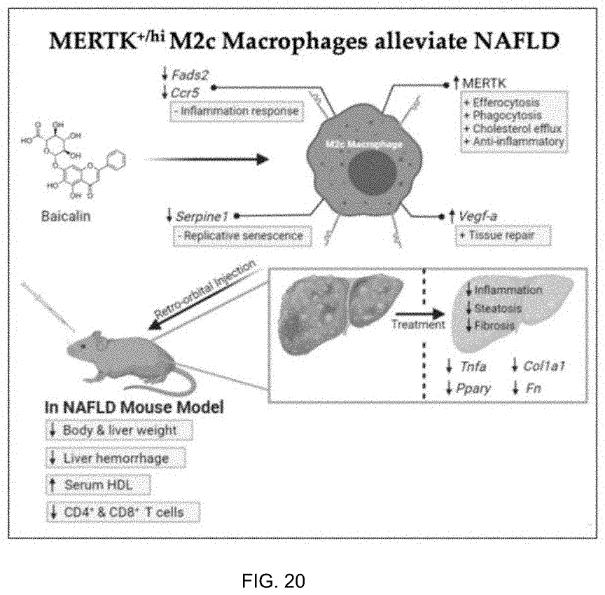

As illustrated in FIG. 20, the treatment with baicalin-induced MERTK^{+/hi} M2 macrophages may alleviate NAFLD through a possible mechanism involving the upregulation of serum high-density lipoprotein cholesterol (beneficial cholesterol) level and the downregulation of NAFLD-associated genes in the liver, including pro-fibrotic COL1A1, pro-fibrotic FN, and lipid metabolism regulator PPARγ.

Possible Application in Industry

Based on the results of the above examples, the invention provides a method of treating imbalance of plasma lipids level in a subject in need, comprising the step of administering a therapeutically effective amount of baicalin induced MERTK-expressing M2C macrophages, wherein the treatment of imbalance of plasma lipids level comprises increasing proportion of high-density lipoprotein cholesterol in plasma lipids. The invention also provides a method of treating fatty liver and nephritis in a subject in need, comprising the step of administering a therapeutically effective amount of baicalin induced MERTK-expressing M2C macrophages.

Biological Deposit

The M2C macrophages are deposited in CCTCC (China Center for Type Culture Collection) with accession number C2017269.

This application contains references to amino acid sequences and/or nucleic acid sequences which have been submitted herewith as the sequence listing text file. The aforementioned sequence listing is hereby incorporated by reference in its entirety pursuant to 37 C.F.R. § 1.52(e).

The patent or application file contains at least one drawing executed in color. Copies of this patent or patent application publication with color drawing(s) will be provided by the Office upon request and payment of the necessary fee.

What is claimed is:

1. A method of treating imbalance of plasma lipids level in a subject in need, comprising the step of administering a therapeutically effective amount of M2C macrophages, wherein the M2C macrophages is MERTK-expressing M2C macrophages prepared by:
isolating mononuclear cells from bone marrow or peripheral blood;
inducing the mononuclear cells to differentiate into M2 macrophages by macrophage colony stimulating factor (M-CSF); and
inducing the M2 macrophages to polarize into MERTK-expressing M2C macrophages by baicalin.

2. The method of claim 1, wherein the treatment of imbalance of plasma lipids level comprises increasing proportion of high-density lipoprotein cholesterol in plasma lipids.

3. The method of claim 1, wherein the M2C macrophages are macrophage cell lines deposited in CCTCC (China Center for Type Culture Collection) with accession number C2017269.

4. A method of treating fatty liver disease in a subject in need, comprising the step of administering a therapeutically effective amount of M2C macrophages, wherein the M2C macrophages is MERTK-expressing M2C macrophages prepared by:
isolating mononuclear cells from bone marrow or peripheral blood;
inducing the mononuclear cells to differentiate into M2 macrophages by macrophage colony stimulating factor (M-CSF); and
inducing the M2 macrophages to polarize into MERTK-expressing M2C macrophages by baicalin.

5. The method of claim 4, wherein the M2C macrophages are deposited in CCTCC (China Center for Type Culture Collection) with accession number C2017269.

6. The method of claim 4, wherein the treatment of fatty liver comprises at least one of reducing microvesicular fatty liver and reducing bridging fibrosis of liver.

7. The method of claim 4, wherein the treatment of fatty liver disease comprises downregulation of non-alcoholic fatty liver disease associated genes in the liver, and the non-alcoholic fatty liver disease associated genes is one or more selected from the group consisting of collagen type 1 alpha (COL1A1), fibronectin (FN), and peroxisome proliferator-activated receptor-gamma (PPARγ).
